SEQUENCE LISTING (3 sequences)

SEQ ID NO: 1 - DNA, 18 bp, synthetic construct
  source 1..18: mol_type = other DNA
aggtttggggaagagaca

SEQ ID NO: 2 - DNA, 18 bp, synthetic construct
  source 1..18: mol_type = other DNA
aggtttggggaggagaca

SEQ ID NO: 3 - DNA, 18 bp, synthetic construct
  source 1..18: mol_type = other DNA
aggtctggggtagagaca

(19) United States
(12) Patent Application Publication
(10) Pub. No.: US 2024/0409985 A1
(43) Pub. Date: Dec. 12, 2024

(54) NUCLEIC ACID HYBRIDIZATION CASCADE ENHANCED BY DROPLET EVAPORATION

(71) Applicant: University of Rochester, Rochester, NY (US)

(72) Inventors: Benjamin L. Miller, Rochester, NY (US); Jeffrey W. Beard, Rochester, NY (US)

(73) Assignee: University of Rochester, Rochester, NY (US)

(21) Appl. No.: 18/698,128

(22) PCT Filed: Sep. 15, 2022
(86) PCT No.: PCT/US2022/076449

§ 371 Date: Apr. 3, 2024

Related U.S. Application Data
(60) Provisional application No. 63/253,722, filed on Oct. 8, 2021.

(51) Int. Cl. C12Q 1/6834; C12Q 1/70

(52) U.S. Cl. CPC C12Q 1/6834; C12Q 1/703

(57) ABSTRACT

The present disclosure relates to novel methods for detection of nucleic acid sequences.

### CROSS REFERENCE TO RELATED APPLICATION

This application claims priority to U.S. Provisional Application No. 63/253,722 filed on Oct. 8, 2021. The content of the application is incorporated herein by reference in its entirety.

### GOVERNMENT INTERESTS

This invention was made with government support under subaward No. 60058448 to prime No. U54 EB027049 by the National Institutes of Health. The government has certain rights in the invention.

### FIELD OF THE INVENTION

This disclosure relates to the fields of molecular biology and diagnostics, such as nucleic acid-based detections.

### BACKGROUND

Nucleic acid-based detection methods are used to detect a particular nucleic acid sequence and thereby to detect and identify a particular species or subspecies of organism (e.g., a pathogen) or physiological/pathological status of cells (e.g., cancerous cells). From cancer screenings to acute viral infection diagnosis, sensitive and accurate detection of nucleic acid sequences plays an important role. The most widely used assays for nucleic acid detection, such as polymerase chain reaction (PCR) and loop-mediated isothermal amplification (LAMP), rely on environmentally sensitive and expensive enzymes that are difficult to maintain in point-of-care (POC) applications. Enzyme-free nucleic acid amplification strategies, such as those based on DNA hairpin assemblies, are becoming increasingly attractive in biological sensors due to their ability to significantly amplify a signal from a single nucleic acid target. However, the enzyme-based nucleic amplification assays are still far more sensitive than DNA hairpin assemblies. Attempts to improve sensitivity of DNA hairpin copolymer assemblies include incorporation of DNAzymes,¹ nucleases,² enzyme-mediated nucleic acid enhancement,³ and gold nanoparticles.^{4,5} However each of these approaches requires addition of an extra element to the reaction, increasing the overall complexity of the assay workflow, assay cost, and a longer reaction time. Thus, there is a need for diagnostics that are rapid, simple, and more cost-effective.

### SUMMARY

This disclosure addresses the need mentioned above in a number of aspects.

In one aspect, this disclosure provides a method for detecting a target nucleic acid in a test sample. The method comprises (i) providing a substrate having a reaction area on which is deposited with the test sample and a detection solution to form a reaction droplet, said detection solution comprising a detection agent having a sequence that is complementary to a strand of the target nucleic acid; (ii) incubating the reaction droplet under conditions allowing evaporation thereof, and (iii) detecting a hybridization complex of the target nucleic acid and the sequence in the reaction droplet. A presence of the hybridization complex indicates presence of the target nucleic acid in the test sample.

In the method described above, the providing step may comprise (a) placing a sample droplet comprising the test sample onto the reaction area and (b) contacting the reaction area or the test sample thereon with the detection solution. The method may further comprise drying (e.g., in a drying chamber) the sample droplet on the reaction area before the contacting step.

In the method described above, the reaction area may be hydrophilic or hydrophobic. It can be surrounded by a hydrophobic area. In one embodiment, the reaction area or the substrate may be surrounded by a non-permeable boundary. The reaction area or the substrate may comprise a material selected from the group consisting of glass, plastic, metal, silicon, and paper. In some embodiments, the substrate comprises a structure of a pillar or a pedestal having a circular top that serves as the reaction area. The circular top may have a diameter of about 0.5 μm to 4 mm (e.g., about 1 μm to about 3 mm, about 100 μm to about 2 mm, or about 500 μm to about 1 mm).

In the method described above, the reaction droplet or the sample droplet can be about 1-25 μl (e.g., about 2-20 μl, about 3-15 μl, or about 4-10 μl).

In some embodiments, the detection solution may comprise a hybridization chain reaction (HCR) mixture as described in details below.

The detection solution may comprise about 15 mM to about 800 mM (e.g., about 20 mM to 500 mM or about 50 mM to 200 mM) concentration of any combination of Na⁺ and K⁺ ions. The detection solution may comprise one or more of about 0.1× to about 5.0×SSC (e.g., about 0.1× to about 2×, about 0.2× to 1.0×, or about 0.25× to 0.5×), about 1 mM to about 80 mM, such as 1 mM to about 15 mM, (e.g., about 2 mM to 15 mM, about 5 mM to 10 mM) Mg²⁺ or Zn²⁺, PBS, TBS, a detergent, glycerol, and sucrose.

The conditions mentioned above may include one or more of the following: a humidity of about 5% to about 95% (e.g., about 10% to about 80%, about 20% to about 50%, or about 30% to about 40%), a temperature of about 10° C. to about 50° C. (e.g., about 15° C. to about 45° C., about 20° C. to about 40° C., or about 30° C. to about 40° C.), and a time duration of about 10 to about 300 minutes (e.g., about 30 to about 240 minutes, about 60 to about 180 minutes, or about 90 to about 120 minutes).

The target nucleic acid can be a DNA or an RNA. In one embodiment, the target nucleic acid is a nucleic acid of a pathogen, such as a virus e.g., HIV, Dengue, SARS-CoV-2, or Ebola. Non-limiting examples of the virus include Norwalk, Rotavirus, Poliovirus, Ebola virus, Marburg virus, Lassa virus, Hantavirus, Rabies, Influenza, Yellow fever virus, Coronavirus, SARS, SARS-CoV-2, West Nile virus, Hepatitis A, C (HCV) and E virus, Dengue fever virus, toga (e.g., Rubella), Rhabdo (e.g., Rabies and VSV), Picorna (Polio and Rhinovirus), Myxo (e.g., influenza), retro (e.g., HIV, HTLV), bunya, corona and reoviruses which have profound effects on human health, including viroid like viruses such as hepatitis D virus and plant RNA viruses and viroids such as Tobus-, Luteo-, Tobamo-, Potex-, Tobra-, Como-, Nepo-, Almo-, Cucumo-, Bromo-, and Ilar-viruses. The test sample described above may comprise a blood sample, a sputum sample, a urine sample, a urinary swab sample, or a saliva sample.

In another aspect, this disclosure additionally provides a kit comprising one or more of the substrate and the detection solution described above. The kit may further comprise one or more probes having a sequence that is complementary to a target sequence. In one embodiment, the kit includes an HCR reaction mixture as described herein.

The details of one or more embodiments of the disclosure are set forth in the description below. Other features, objectives, and advantages of the disclosure will be apparent from the description and from the claims.

### BRIEF DESCRIPTION OF THE DRAWINGS

FIG. 1 is a diagram showing schematic of hybridization chain reaction (HCR) using Forster Resonance Energy Transfer (FRET) for fluorescence detection of a target trigger sequence or initiator sequence. H1 has a complementary region to the initiator sequence, causing it to unfold from its secondary structure and hybridize with the initiator, exposing a complementary sequence to H2. H2 hybridizes with H1, exposing a complementary sequence to H1, thus continuing the cascade. The intensity of the H2 acceptor fluorophore is dependent on the length of the H1/H2 HCR product. The reaction can be incubated in a sessile droplet (bottom).

FIGS. 2A and 2B are photographs of agarose gels showing HCR products at varying trigger concentrations incubated in (FIG. 2A) cuvettes at room temperature for 2.5 hours and (FIG. 2B) in 5 μL sessile droplets till dry (˜1.5 hours in ambient conditions).

FIGS. 3A, 3B, and 3C are photographs showing (FIG. 3A) custom 3D printed sessile droplet pedestals with 3 μL droplets of HCR mix, fluorescence microscope images of (FIG. 3B) 0 nM and (FIG. 3C) 1 nM trigger concentrations of dried HCR-FRET sessile droplets.

FIG. 4 is a photograph showing direct comparison of sessile droplet enhanced HCR (S) to cuvette incubated HCR (L) using 0.25×SSC buffer with 10 mM MgCl₂. Trigger concentrations decrease left to right from 100 nM trigger to 0 nM trigger.

FIG. 5 is a diagram showing relative FRET intensities of liquid cuvette incubated HCR and sessile enhanced HCR at 0 nM trigger and 100 nM trigger concentrations using 0.25×SSC buffer with 10 mM MgCl₂.

FIG. 6 is a photograph showing sessile droplet enhanced hybridization chain reaction incubated in buffers with varying concentrations of sodium and magnesium. Each box shows a comparison of HCR product between a trigger concentration of 0 pM and 500 pM with their corresponding buffer.

FIGS. 7A and 7B are photographs showing an example of a sessile droplet experimental device and setup. FIG. 7A shows three PDMS rings containing 1.5 mm diameter pedestals surrounded by desiccant. The lid with windows is placed over the pedestals for controlled evaporation. FIG. 7B shows an enlarged image of one ring containing six HCR sessile droplets suspended on the pedestals.

FIG. 8A is a set of photographs of agarose gels showing improved hybridization of HCR through sessile droplet incubation when compared to traditional cuvette incubation.

FIG. 8B and FIG. 8C show spectrofluorometer data showing the normalized FRET intensities of HCR product from synthetic proviral DNA target (FIG. 8B) and synthetic viral RNA target (FIG. 8C), when incubated in both a sessile droplet and cuvette.

FIG. 9 shows agarose gel showing HCR incubated against synthetic DNA trigger in two different conditions, a wet condition (W) and a dry condition (D).

FIG. 10A shows HCR sessile droplet incubations with 0, 1 and 2 mismatches in the synthetic DNA trigger sequences.

FIG. 10B shows fluorometric FRET results of HCR incubated in a sessile droplet with synthetic trigger DNA and a 5:1 mass ratio of salmon DNA to synthetic trigger DNA, demonstrating a high resistance to off target hybridization.

FIG. 11A shows FRET intensities of sessile droplet HCR product when incubated in buffers with differing ion concentrations.

FIG. 11B shows experimental melt temperature of the H1 hairpin at different sodium concentrations.

FIG. 12A shows 1.5 mm diameter pads that were cut and adhered to PDMS pedestals to allow for sessile droplet formation on the hydrophilic material. Target nucleic acids were dried onto the paper disks.

FIG. 12B shows that 5 μL of HCR solution (H1 and H2) were applied to each pedestal as a sessile droplet and allowed to dry completely.

FIG. 12C shows that dried paper was then imaged using a fluorescence microscope with a FRET cube. Image intensities were analyzed using ImageJ. P values were calculated using a two-tailed Student's t-test in JMP Pro 16.

FIG. 12D and FIG. 12E show sessile droplet HCR FRET intensities detecting synthetic HIV RNA (FIG. 12D) and proviral DNA (FIG. 12E) directly on filter paper (Fusion 5, Cytiva).

### DETAILED DESCRIPTION OF THE INVENTION

This disclosure relates to molecular biology and diagnostics, such as nucleic acid-based detection methods, devices, and systems. The diagnostic methods described herein are simple to perform, do not require instrumentation or expensive enzymes, are inexpensive to manufacture, are stable over a variety of environmental conditions, and can be readily interpreted by an unskilled end-user. As most conventional nucleic acid detection tests suffer from extreme sensitivity to thermal storage conditions and poor performance at low target concentrations, the methods, devices, and systems described herein have considerable advantages over the conventional approaches and are suitable for point-of-care (POC) applications, particularly as rapid diagnostics for non-industrialized regions around the world.

Certain aspects of this disclosure are based, at least in part, on an unexpected discovery that the efficiency of hybridization of nucleic acid structures can be enhanced by conducting the reaction in a low volume droplet (e.g., picoliters to microliters) of solution, referred to as a sessile droplet, exposed to atmospheric conditions and/or allowed to evaporate. Sessile droplets described herein provide benefits of internal currents and decreasing volume when evaporating that allow for enhanced molecular interactions. The Marangoni Effect (i.e., evaporation-induced currents created by thermodynamic variances throughout the droplet) is believed to create a self-mixing environment within the sessile droplet, increasing molecular interactions in an otherwise diffusion limited reaction.^{7,8}

Although sessile droplets have been used for enhancing bimolecular interactions, they were not employed for a complex assembly process such as HCR. For instance, the use of sessile droplets has been proven to increase reaction kinetics and decrease overall reaction time in the context of enzyme-driven colorimetric assays.⁶ Thus far, however, this has been limited to simple interactions between two molecules. The methods, devices, and systems disclosed in this disclosure leverage the benefits of sessile droplet evaporation to increase the copolymer hybridization of nucleic acid nanostructures and thereby improve capabilities for enzyme-free nucleic acid detection.

### Substrates and Devices

The detection methods described herein involve conducting a nucleic acid hybridization reaction in a sessile droplet residing on a substrate. The droplet is exposed to atmospheric conditions and allowed to evaporate.

Utilizing evaporation allows concentration of detection targets to greatly shorten the reaction time and enhance the detection sensitivity. In addition, upon evaporation, natural convection arising from Marangoni currents mixes solutions in the droplet, which speeds up assay reactions, decreases assay times, and increases limits of detection. Differential evaporation rates resulted in movement within a sessile droplet. Depending on the content of the droplet (e.g., salt), and various environmental factors affecting evaporation (e.g., temperature differential), the evaporation may produce primary radial flow or Marangoni flow. Altering these controlling factors to produce one or the other is a primary design consideration in low resource assays relying on drop deposition. Accordingly, design parameters including substrate material and solution components may be optimized to promote the Marangoni flow. Depending on the design considerations, this flow pattern can be axisymmetric and directed radially outward along the air liquid interface and radially inward along the substrate, or can be directed in the opposite direction.

The surface tension gradient from which Marangoni flow fields arise is thought to be caused by a temperature gradient that arises from cooling effects caused by the non-uniform evaporative flux along the drop surface. The evaporation rate of a drop is greatest at the contact line due to the proximal location of ambient, unsaturated gas resulting in non-uniform evaporation along the air-liquid interface (Deegan et al., Physical Review E, 62, (1), 756-765, 2000.). The extent to which non-uniform evaporative cooling effects result in a temperature gradient along the drop surface is determined in part by the rate of heat transfer from the isothermal substrate to the air-liquid interface (Ristenpart et al., Physical Review Letters, 99, (23), 2007). These heat transfer rates are, in part, a function of both the drop height as well as the thermal conductivities of the substrate and liquid.

If the thermal conductivity of the substrate is sufficiently low, evaporative cooling dominates and causes the lowest temperature to occur at the contact line. Conversely, a highly thermally conductive substrate promotes sufficient heat transfer at the contact line to overcome evaporative cooling effects resulting in the greatest temperature at the drop edge and lowest at the center. These temperature gradients cause surface tension gradients which in turn drive the Marangoni flow. The drop is coolest at the contact line if the substrate has a thermal conductivity less than, e.g., 1.45 times that of the liquid causing fluid to flow radially outward along the air-liquid interface and radially inward to the center of the droplet along the substrate interface (Ristenpart et al., Physical Review Letters, 99, (23), 2007 and U.S. patent Ser. No. 10/101,323). That is, the fluid flows along the substrate toward the center of the drop then turns toward the air-liquid interface and flows in the direction of the contact line along the drop surface. If the substrate has a thermal conductivity greater than, e.g., 2 times that of the liquid, the flow direction is reversed. Moreover, these Marangoni flow fields are axisymmetric around the drop center resulting in a toroidal geometry when viewed from above the drop.

Accordingly, substrates that can be used herein may include those having suitable thermal conductivities so that temperature gradients can cause sufficient surface tension gradients which in turn drive the Marangoni flow.

The substrate can be permeable or non-permeable to water or an aqueous solution. It has a reaction area adapted to receive a nucleic acid, or a sample composition containing nucleic acid, or a solution for hybridization reaction. In some embodiments, this reaction area is flat and non-permeable to water or an aqueous solution. In some other embodiments, a substrate permeable to water or an aqueous solution can be used as long as enough liquid can saturate the permeable substrate and produce a sessile droplet on the surface of that substrate. In that case, initial capillary forces can pull some nucleic acid (such as targets and DNA probes) material into the permeable substrate, but shortly after, evaporative forces (not necessarily Marangoni forces) in the droplet can theoretically pull the material out of the substrate. Thin permeable layers such as a section of Fusion 5 filter paper can support a sessile droplet and can be imaged using fluorescence microscopy in a similar method to a non-porous, hydrophobic surface. Thicker permeable substrates, such as TFN—blood spot transport paper—can also be usable under the previously described conditions and then the reaction can be eluted from the permeable dot and analyzed using any suitable methods (e.g., fluorescence microscopy, gel electrophoresis, etc.).

The substrate can be made of, or coated with, virtually any type of material that provides the necessary fluid dynamics and attachment of the sample composition or nucleic acid. Examples of such materials include glass, plastic (e.g., polystyrene), latex, metal, polymers, silica, metal oxides, ceramics, or any other substance suitable for binding to nucleic acids or other material with appropriate thermal conductivity and contact angle (hydrophobic). The area may be derivatized to facilitate the use of different solvent and detection methods. Substrates having high thermal conductivity and/or a high contact angle (hydrophobic) are desired. In some embodiments, the droplet can form a contact angle of 700 or larger.

For example, the nucleic acid reaction area may be formed of glass, plastic, metal, silicon, paper, cloth, woven, or non-woven cellulose substrates. In some embodiments, the reaction area may be formed of paper, such as commercially available filter paper (e.g., Whatman paper, nitrocellulose, or fiberglass). In some embodiments, the reaction area may be functionalized so as to facilitate attachment of nucleic acid. For example, the nucleic acid-receiving area may be functionalized to be positively charged with, e.g., chitosan or polyethyleneimine (PEI). The term “functionalized” or “chemically functionalized,” as used herein, means addition of functional groups onto the surface of a material by chemical reaction(s). As will be readily appreciated by a person skilled in the art, functionalization can be employed for surface modification of materials in order to achieve desired surface properties, such as biocompatibility, wettability, and so on.

The substrate or the reaction area thereon can be of various geometric shapes and sizes. For example, the substrate or the reaction area may have a circular, semi-circular, triangular, square, rectangular, pentagonal, or hexagonal shape. In some embodiments, the substrate or the reaction area has a circular shape and may have a diameter, e.g., from about 0.5 μm to about 4 mm (e.g., about 1 μm to about 2 mm, about 2 μm to about 1 mm, and about 5 μm to about 500 μm). In some embodiments, the substrate or the reaction area is provided in the form of microarray suitable for high throughput assays.

The amount of a fluid sample that is applied to the substrate or reaction area may vary, so long as it is sufficient to provide for the desired volume and operability of the assay. In some embodiments, the substrate or reaction area is adapted for a small volume of sample, e.g., from about 1 μL to about 25 μL (e.g., about 2 μL to about 20 μL, about 3 μL to about 15 μL, and about 10 μL).

The substrate described above may be included in a biosensor device for enriching and detecting biomarker molecules, such as nucleic acids. The device may comprise the substrate and optionally other components such as a support holding the substrate, a hydrophobic surface surrounding the substrate, or a chamber housing the substrate. For example, such a device may comprise a support; one or more substrates disposed on the support; one or more reaction areas disposed on the substrates; a hydrophobic surface surrounding substrate.

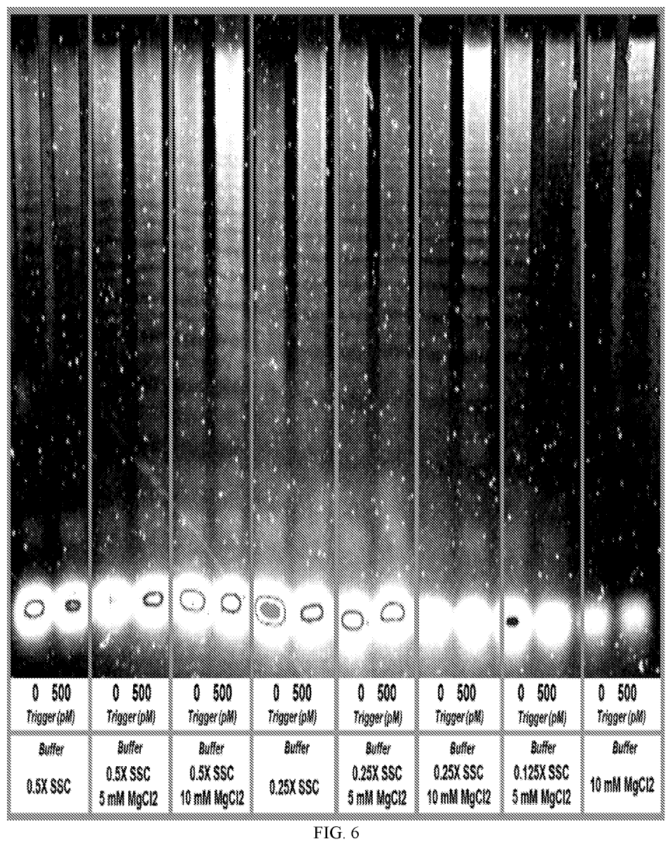

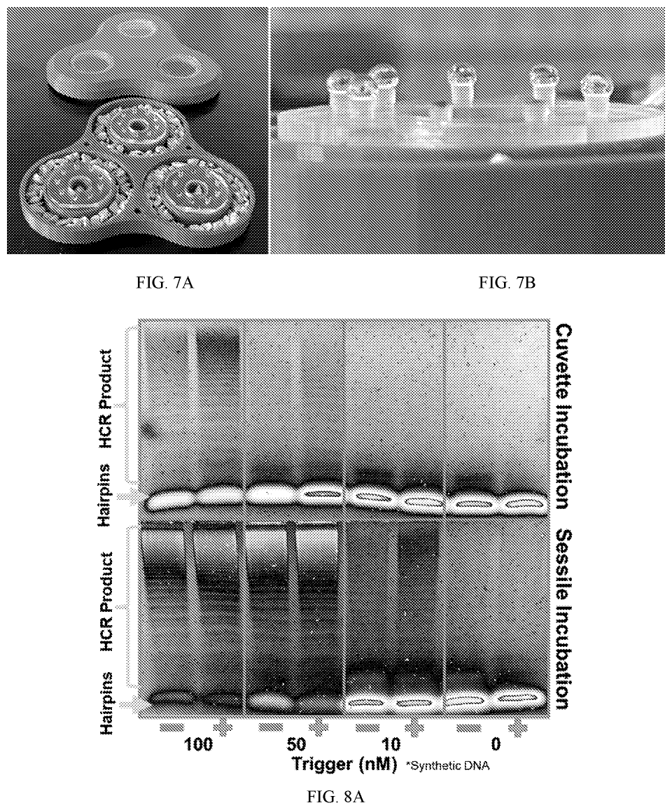

The device may also include a chamber to house all the other components for controlling a reaction between the target biomarker molecules and the detection agents. Shown in FIG. 7A is an exemplary device. The device includes a housing for three PDMS ring shaped supports. On each support are nine pedestals (1.5 mm diameter) surrounded by desiccant. The lid with windows is placed over the pedestals for controlled evaporation. FIG. 7B shows an enlarged image of one ring containing six HCR sessile droplets suspended on the pedestals.

The reaction area can be structured to receive a test sample or/and detection solution. The reaction area can also be structured to anchor one or more detection agents (e.g., probes or other agents) for detecting molecules of a target biomarker. The reaction area can also be structured to attract an array of droplets of a biomarker solution that includes the target biomarker molecules. The hydrophobic surface surrounding the reaction area may include nanostructures having rough surfaces to enrich the target biomarker molecules onto the reaction area by enhancing evaporation of the array of droplets leading to an enriched array of droplets with an increased concentration of the target biomarker molecules compared to before evaporation.

In some embodiments, the substrate may include a reaction area that is surrounded by a non-permeable boundary. In some embodiments, the reaction area can be hydrophilic and surrounded by a hydrophobic area. For example, the substrate may include hydrophilic (e.g., SiO₂, metal, dielectrics, or other hydrophilic materials) patterns surrounded by micro- or nano-patterned, hydrophobic or super-hydrophobic, black silicon surface.

A super-hydrophobic surface can produce a large contact angle of well over 90 or 150 degrees for droplets of aqueous solution. In the case of black silicon and nanostructured polydimethylsiloxane (PDMS) surfaces, the contact angle of water droplets can be greater than 150 degrees. In one embodiment to achieve super-hydrophobic surface, the device can include an array of hydrophilic micro-islands or microarray of hydrophilic islands surrounded by super-hydrophobic black silicon fabricated on a Si wafer. To form black silicon, an exemplary Bosch etching process can be employed in a reactive ion etcher in the manner described in, e.g., US 20160258020, the disclosures of which are incorporated by reference. The etch process includes alternating cycles of etching (SF₆) and passivation (C₄F₈) to form nanopillar structures. For areas that are protected by lithographically defined SiO₂ patterns, no etching or passivation occurs so the hydrophilic properties are preserved after the black silicon process.

In another embodiment to achieve super-hydrophobic surface, the device can include an array of hydrophilic areas, islands, pedestals, or pillars surrounded by super-hydrophobic PDMS areas formed using 3-D printing or nanoimprinting or hot embossing process in the manner described in, e.g., US 20160258020, the disclosures of which are incorporated by reference.

Nucleic acid-containing samples can be enriched and hybridized on such a device via rapid evaporation followed by self-assembled droplet (e.g., microliter, nanoliter, or less) formation. Within the droplets, the hybridization reaction proceeds to completion rapidly due to the reduced volume, resulting in accelerated hybridization time. Moreover, the precise volume control offered by self-assembled droplet (e.g., microliter, nanoliter, or less) array allows for a wide range of control over enrichment ratio, resulting in higher sensitivity and extended dynamic range.

### Detecting Nucleic Acids

Various methods for detecting nucleic acids are known in that art and can be used herein. In some embodiments, nucleic acid copolymer assemblies such as those created by hybridization chain reaction (HCR) can be used to significantly amplify signal from a target nucleic acid without the use of enzymes or thermocycling, making them ideal for point-of-care diagnostics. Conducting the reaction in a sessile droplet, which is exposed to atmospheric conditions and allowed to evaporate, further enhances the efficiency of the copolymer hybridization.

### HCR

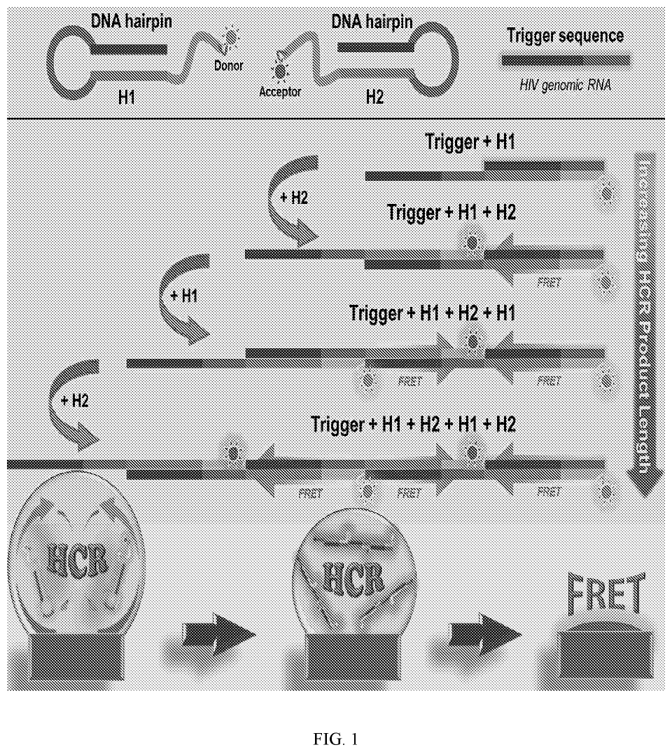

Described herein are novel and rapid methods for enhancing hybridization kinetics for increased nucleic acid detection sensitivity without adding extra assay reagents or reaction time by forming hybridization within sessile droplets. In some embodiments, detecting one or more nucleic acids in a biological sample is carried out based on a nonenzymatic method of nucleic acid amplification termed a hybridization chain reaction as shown in FIG. 1.

HCR, a nucleic acid amplification technique, leverages naturally occurring DNA self-assembly to form extended notched dsDNA assemblies. HCR does not require any enzymes and can operate isothermally. Various formats of HCR are known in the art and can be used in the methods described herein. See, for example, Dirks, R. and Pierce, N. Proc. Natl. Acad. Sci. USA 101(43): 15275-15278 (2004), U.S. Pat. Nos. 8,105,778 and 8,507,204, US Pat. Pub Nos. 201902186080 and 20180010166, each of which is incorporated herein by reference in its entirety.

HCR can involve an initiator sequence, and two or more strategically designed metastable hairpin monomers (e.g., H1 and H2 shown in FIG. 1), whose secondary structures form meta-stable hairpins. The hairpin monomers each have at least one single-stranded toehold, a single-stranded loop, and a double-stranded stem. The energy to drive the self-assembly cascade is stored in the single-stranded loop and toehold segments of the hairpins. Each monomer is caught in a kinetic trap, preventing the system from rapidly equilibrating. That is, pairs of monomers are unable to hybridize with each other in the absence of an initiator. Introduction of an initiator strand causes the monomers to undergo a chain reaction of hybridization events to form a nicked double-stranded polymer. HCR can be used, for example, to detect the presence of an analyte of interest in a sample by detecting the analyte with a probe that carries an HCR initiator, which in turn triggers HCR signal amplification. HCR signal amplification makes it possible to increase the signal-to-background ratio for molecular detection and imaging applications by boosting the signal above the background arising from the sample.

As used herein, “monomers” are individual nucleic acid oligomers. Typically, at least two monomers are used in hybridization chain reactions, although three, four, five, six or more monomers may be used. Typically each monomer comprises at least one region that is complementary to at least one other monomer being used for the HCR reaction.

An “initiator” or “trigger” is a molecule that is able to initiate the polymerization of monomers. Example initiators may include a nucleic acid region that is complementary to the initiator complement region of an HCR monomer. An initiator may be, for example, an analyte of interest, or a nucleic acid that is able to contact the first monomer (e.g., HI in FIG. 1) only in the presence of an analyte of interest, as discussed in more detail below. For example, an initiator can be a nucleic acid to be detected itself (e.g., a viral RNA).

As shown in FIG. 1, H1 has a region complementary to an initiator (trigger) sequence, causing it to unfold from its secondary structure and hybridize with the initiator, exposing a complementary sequence to H2. H2 hybridizes with H1, exposing a complementary sequence to H1, thus continuing the cascade (FIG. 1). In the case of FRET experiments described herein, HI can be conjugated with a donor fluorophore and H2 can be conjugated with an acceptor fluorophore. The intensity of the H2 acceptor fluorophore is dependent on the length of the resulting H1-H2 HCR product.

In one example, a single copy of a target nucleic acid functions as the initiator sequence and is contacted with a reaction solution or reaction mixture containing a HCR probe set, which include two types of DNA hairpin probes, H1 and H2. Once contacted, the target nucleic acid/initiator sequence triggers opening of a first H1 DNA hairpin via duplex formation, which in turn triggers the opening of a first H2 DNA hairpin. This opened H2 then binds to a region of a second H1, opens this second H1, and the cascade continues. Each hairpin can be labeled at its terminus or termini with a donor fluorophore or an acceptor fluorophore as described above; thus, binding and opening of each hairpin, as the cascade progresses, causes FRET and an increase in signal for quantification by an imaging device, e.g., a smartphone camera, whereby resonance energy is transferred from the electron of a donor fluorophore to an acceptor fluorophore and emitted as a photon with a longer wavelength than a photon emitted by the donor fluorophore A.

In some embodiments, the reaction solution/mixture comprises an HCR mixture. In some embodiments, the reaction solution/mixture comprises one or more probes (e.g., HCR probe set) having a sequence that is complementary to a target sequence of the nucleic acid.

An “HCR probe set” or “HCR initiator/hairpin set” may include one or more initiator strands of nucleic acid together with one or more metastable HCR monomers, such as nucleic acid hairpins, that together are capable of forming the hybridization chain reaction polymer. HCR probes may be synthesized using standard methods, such as chemical nucleic acid synthesis, including commercial sources such as Integrated DNA Technologies (IDT, Coralville, Iowa), W. M. Keck Foundation Oligo Synthesis Resource (New Haven, Connecticut), or Molecular Instruments (Pasadena, California). Alternatively, the HCR probes may be synthesized and/or amplified using standard enzymatic methods, such as PCR followed by lambda exonuclease digestion of one strand to yield ssDNA, (see Current Protocols in Molecular Biology (2014): 14-23, hereby incorporated by reference in its entirety) or in vitro transcription followed by reverse transcription to yield ssDNA (see, e.g., Chen et al., Science 348:6233 (2015): aaa6090, hereby incorporated by reference in its entirety).

In one embodiment, to enhance enzyme-free nucleic acid detection for rapid diagnostic applications, a target nucleic acid sequence (“target”), such as mRNA or a sequence of genomic DNA or RNA, is applied to and/or captured on a reaction zone composed of either a hydrophilic circular pad surrounded by hydrophobic material, or a circular post. The target becomes enriched after being applied to the reaction zone as the droplet volume decreases due to evaporation. The target is dried or mostly dried on the reaction zone before a sessile droplet of DNA hairpin reaction solution/mixture is applied. Evaporation of the reaction solution/mixture simultaneously causes internal mixing of the reaction solution/mixture and increases reaction concentration due to decreasing droplet volume. More H1 and H2 hairpins are able to hybridize during the evaporation process, increasing the HCR product and subsequently the signal output of the assay. In the instance of examples described herein, the signal output is the intensity of the acceptor fluorophore of the H1/H2 FRET pair.

In certain embodiments, the evaporation process of the droplets may be facilitated by the hydrophobic or superhydrophobic surface and resulting droplets (e.g., nanoliter volume or less) can be encapsulated by water-immiscible-liquid (e.g., oil) drops to form stable reaction chamber (e.g., nano or microliter volume) in the manner described in US 20160258020, the disclosures of which are incorporated by reference.

The test sample, the reaction solution/mixture, or a mixture of both may be applied to the reaction area or site using any convenient protocol, e.g., via a dropper, pipette, syringe, and the like.

In one example, the test sample is mixed with the reaction solution/mixture before being applied to the reaction area. In another example, the test sample may be applied to the reaction area or site concurrently with, before, or after applying reaction solution/mixture to the reaction area.

In addition, a test ample or the reaction solution/mixture may be applied to the area along with any suitable liquid, e.g., buffer, to provide for adequate reaction or hybridization conditions. Examples of a suitable liquid may include, without limitation, buffers. Examples of buffers include, but are not limited to tris, tricine, SSC, MOPS, HEPES, PIPES, MES, PBS, TBS, and the like.

In one example, the suitable liquid may be mixed with the test sample or the reaction solution/mixture before being applied to the reaction area. In another example, the suitable liquid may be applied to the reaction area or site concurrently with, before, or after applying the sample.

Given the clear advantages of HCR in this droplet format, it has broad utility in molecular diagnostics as an alternative to PCR. In one embodiment, this format of HCR may be used for POC or at-home viral load monitoring for a pathogen, such as HIV. Of global importance, the sessile droplet assay format may have particular utility in resource-limited environments such as sub-Saharan Africa, where cost, environmental conditions, and assay complexity make standard enzyme-based nucleic acid amplification strategies challenging.

Accordingly, also provided in this disclosure is a method for identifying a patient having a viral infection or suspected of having a viral infection. The method may include: (i) obtaining a sample (e.g., blood sample) for the patient; (ii) determining a level of the viral load (e.g., a level of HIV RNA) in the blood sample by a method described above; (iii) comparing the determined level of the viral load with a control level and determining whether the determined level is elevated as compared to the control level; and (iv) determining that the patient a viral infection if the determined level of the viral load is elevated as compared to the control level.

The terms “patient,” “individual,” and “subject,” are used interchangeably and generally refer to any living organism to which the disclosed methodology is utilized to obtain a bodily fluid sample in order to perform a diagnostic or monitoring method described herein. A patient can be an animal, such as a human. A patient may also be a domesticated animal or a farm animal. A “patient” or “individual” may also be referred to as a subject.

As used herein, a “control” level of a pathogen, e.g., a viral load, refers, in some embodiments, to a level of pathogen obtained from a sample obtained from one or more individuals who do not suffer from a disease or disorder that is of interest in the investigation, e.g., viral infection such as HIV infection. The level may be measured on an individual-by-individual basis or on an aggregate basis such as an average. A “control” level can also be determined by analysis of a population of individuals who have the disease or disorder but are not experiencing an acute phase of the disease or disorder. A “control” sample may be used to obtain such a “control” level. A control sample may be obtained from one or more individuals who do not suffer from a disease or disorder that is of interest in the investigation.

A control sample can also be obtained from a population of individuals who have the disease or disorder but are not experiencing an acute phase of the disease or disorder. In some embodiments, a control level is from the same individual for whom a diagnosis is sought or whose condition is being monitored, but is obtained at a different time. In certain embodiments, a control level or sample can refer to a level or sample obtained from the same patient at an earlier time, e.g., weeks, months, or years earlier.

As used herein, “the determined level is elevated as compared to the control level” refers to a positive change in value from the control level.

### Detection

One of the important advantages of the assays disclosed herein is the use of simple and inexpensive detection methods that do not require sophisticated equipment or trained personnel. Yet, the assays also are amenable to such more sophisticated procedures, which are within the scope of this disclosure.

The products of HCR can be readily detectable by methods known to one of skill in the art for the detection of nucleic acids, including, for example, agarose gel electrophoresis, polyacrylamide gel electrophoresis, capillary electrophoresis, and gel-filled capillary electrophoresis. As the polymers comprise nucleic acids, they can be visualized by standard techniques, such as staining with ethidium bromide. Other methods also can be suitable including light scattering spectroscopy, such as dynamic light scattering (DLS), viscosity measurement, colorimetric systems, mass spectrometry, and fluorescence and fluorescence polarization spectroscopy. As discussed in more detail, in some methods for in situ imaging and detection, HCR products are fluorescently labeled.

Visible detection is one of the most straightforward detection approaches. Visible detection may rely on a color (presence or absence), color change, luminescence, or fluorescence. The present disclosure contemplates that appearance, color, or light of the product produced from drop evaporation will provide a readily recognizable feature that permits home and field use of the assays with minimal instruction (i.e., package inserts with instructions and diagrams).

In some embodiments, light or optical microscopy can be used. Light or optical microscopy involves passing visible light transmitted through or reflected from the sample through a single or multiple lenses to allow a magnified view of the sample. The resulting image can be detected directly by the eye, imaged on a photographic plate or captured digitally. The single lens with its attachments, or the system of lenses and imaging equipment, along with the appropriate lighting equipment, sample stage and support, makes up the basic light microscope. One example is a digital microscope which uses a CCD camera to focus on the exhibit of interest. The image may be shown on a computer screen since the camera can be attached to it via a USB port. Another is a smart phone equipped with a digital camera. Any suitable imaging microscopy techniques may also be used, including bright field microscopy, dark field microscopy, and fluorescence microscopy.

In some embodiments HCR is monitored by FRET. Certain monomers are labeled with fluorescent dyes so that conformational changes resulting from HCR can be monitored by detecting changes in fluorescence. In one embodiment, one hairpin molecule (e.g., H1 in FIG. 1) is labeled with a donor fluorophore while another hairpin molecule (e.g., H2 in FIG. 1) is labeled with an acceptor fluorophore. Upon polymerization, the donor fluorophore and acceptor fluorophore are aligned in close proximity in the aggregated nucleic acid structure, thereby providing FRET. In this case, the presence of a single initiator is amplified by the chain of fluorescent events caused by HCR. In the context of in situ imaging, the presence of a single target molecule can be amplified by the chain of fluorescence events.

### Samples and Diagnostic Targets

The methods, devices, and systems disclosed herein can be used for enrichment and detection of biomarker molecules, such as DNAs and RNAs, without requiring any amplification steps to increase the copy number of those molecular markers. Such methods, devices and systems are especially attractive to a variety of applications including, but not limited to, gene expression analysis by estimating the copy numbers of DNA or RNA transcripts present in a sample, applications in molecular detection, nucleic acid biomarker quantification for clinical, laboratory and point-of-care diagnostic purposes, development to detect pathogen and infectious disease with direct identification of endogenous sequences, including fragmental microbial genome (DNA) and RNA sequences, capability to quantify miRNA and other forms of circulating epigenetic marker for early diagnosis of disease and injury, and mutational analysis of cancer and hereditary diseases using.

Various samples can be used with the methods disclosed herein. As used herein, the term “sample” refers to anything capable of being analyzed by the methods provided herein. In some embodiments, the sample comprises or is suspected to comprise one or more nucleic acids capable of analysis by the methods. Preferably, the samples comprise nucleic acids (e.g., DNA, RNA, cDNAs, microRNA, mitochondrial DNA, etc.). Samples may be complex samples or mixed samples, which contain nucleic acids comprising multiple different nucleic acid sequences (e.g., host and pathogen nucleic acids; mutant and wild-type species; heterogeneous tumor). Samples may comprise nucleic acids from more than one source (e.g., difference species, different subspecies, etc.), subject, and/or individual. In some embodiments, the methods provided herein optionally comprise purifying the sample or purifying the nucleic acid(s) from the sample. In some embodiments, the sample may contain purified nucleic acid. In some embodiments, a sample is derived from a biological, clinical, environmental, research, forensic, or other source.

In some embodiments, the methods, compositions, systems, and devices disclosed herein make use of samples that include a nucleic acid template. Samples may be derived from any suitable source, and for purposes related to any field, including but not limited to diagnostics, research, forensics, epidemiology, pathology, archaeology, etc. A sample may be biological, environmental, forensic, veterinary, clinical, etc. in origin. Samples may include nucleic acid derived from any suitable source, including eukaryotes, prokaryotes (e.g., infectious bacteria), plants, mammals, humans, non-human primates, canines, felines, bovines, equines, porcines, mice, viruses, etc. Samples may contain, e.g., whole organisms, organs, tissues, cells, organelles (e.g., chloroplasts, mitochondria), synthetic nucleic acid, cell lysate, etc. Nucleic acid present in a sample (e.g., target nucleic acid, template nucleic acid, non-target nucleic acid, contaminant nucleic acid may be of any type, e.g., genomic DNA, RNA, plasmids, bacteriophages, synthetic origin, natural origin, and/or artificial sequences (non-naturally occurring), synthetically-produced but naturally occurring sequences, etc.

Biological specimens may, for example, involve solid material such as feces or tissues (including biopsies), tissue extracts, or fluids, including whole blood, lymphatic fluid, serum, plasma, buccal, sweat, tear, saliva, sputum, cerebrospinal (CSF) fluids, amniotic fluid, seminal fluid, vaginal excretions, serous fluid, synovial fluid, pericardial fluid, peritoneal fluid, pleural fluid, transudates, exudates, cystic fluid, bile, urine, gastric fluids, intestinal fluids, fecal samples, and swabs, aspirates (bone marrow, fine needle) or washes (e.g., oral, nasopharangeal, bronchial, bronchialalveolar, optic, rectal, intestinal, vaginal, epidermal, etc.) and/or other fresh, frozen, cultured, preserved (PAXgene, RNAlater, RNasin, etc.) or archived (formalin fixed paraffin-embedded (FFPE), fixed cell/lymphocyte pellet, etc.) biological specimens. Such samples may be solubilized or diluted, as needed, to perform the assays of the present disclosure. Solvents for use in solubilizing or diluting samples include water, acetone, methanol, toluene, ethanol or others.

Other samples include manufactured, industrial or environmental samples, and may or may not contain living cells or organisms. Such sample may include soil, water, foodstuffs, alcoholic beverages, building products, bulk chemicals or reagents, including drugs. Again, such samples may be solubilized or diluted, as needed, to perform the assays of the present disclosure.

In preferred embodiments, the assays of the present disclosure can be used to detect various infectious agents. Infections refer to any condition in which there is an abnormal collection or population of viable intracellular or extracellular microbes in a subject. Various types of microbes can cause infection, including bacteria, viruses, fungi, and parasites. Detection of nucleic acids associated with these microbes within the scope of this disclosure.

Examples of viruses include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunga viruses, phleboviruses and Nairo viruses); Arenaviridae (hemorrhagic fever viruses); Reoviridae (e.g., reoviruses, orbiviurses and rotaviruses); Bornaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g., African swine fever virus); unclassified viruses (e.g., the agent of delta hepatitis (thought to be a defective satellite of hepatitis B virus), Hepatitis C; Norwalk and related viruses, and astroviruses); and resipiratory syncytial virus (RSV).

Examples of bacteria include, pneumococci, streptococci such as S. pyogenes, S. agalactiae, S. equi, S. canis, S. bovis, S. equinus, S. anginosus, S. sanguis, S. salivarius, S. mitis, S. mutans, other viridans streptococci, peptostreptococci, other related species of streptococci, enterococci such as Enterococcus faecalis, Enterococcus faecium, staphylococci, such as Staphylococcus epidermidis, Staphylocccus aureus, Hemophilus influenzae, pseudomonas species such as Pseudomonas aeruginosa, Pseudomonas pseudomallei, Pseudomonas mallei, brucellas such as Brucella melitensis, Brucella suis, Brucella abortus, Bordetella pertussis, Borellia species, such as Borellia burgedorferi Neisseria meningitidis, Neisseria gonorrhoeae, Moraxella catarrhalis, Corynebacterium diphtheriae, Corynebacterium ulcerans, Corynebacterium pseudotuberculosis, Corynebacterium pseudodiphtheriticum, Corynebacterium urealyticum, Corynebacterium hemolyticum, Corynebacterium equi, etc. Listeria monocytogenes, Nocordia asteroides, Bacteroides species, Actinomycetes species, Treponema pallidum, Leptospirosa species, Haemophilus species, Helicobacter species, including Helicobacter pylori, Treponema species and related organisms. The invention may also be useful against grain negative bacteria such as Klebsiella pneumoniae, Escherichia coli, Proteus, Serratia species, Acinetobacter, Yersinia pestis, Francisella tularensis, Enterobacter species, Bacteroides and Legionella species, Shigella species, Mycobacterium species (e.g., Mycobacterium tuberculosis, Mycobacterium bovis or other mycobacteria infections), Mycobacterium avium complex (MAC), Mycobacterium marinum, Mycobacterium fortuitum, Mycobacterium kansaii, Yersinia infections (e.g., Yersinia pestis, Yersinia enterocolitica or Yersinia pseudotuberculosis) and the like.

Examples of parasitic organisms include Cryptosporidium, Entamoeba, Plasmodium spp., such as Plasmodium falciparum, Plasmodium malariae, Plasmodium ovale, and Plasmodium vivax and Toxoplasma gondii, Giardia, Leishmania, Trypanosoma, Trichomonas, Naegleria, Isospora belli, Trichomonas vaginalis, Wuchereria, Ascaris, Schistosoma species, Cyclospora species, for example, and for Chlamydia trachomatis and other Chlamydia infections such as Chlamydia psittaci, or Chlamydia pneumoniae, for example. Of course, it is understood that the invention may be used on any pathogen against which specific probes (e.g., H1 and H2 hairpin probes as described herein) can be made.

Fungal and other mycotic pathogens (some of which are described in Human Mycoses (1979; Opportunistic Mycoses of Man and Other Animals (1989); and Scrip's Antifungal Report (1992), are also contemplated as a target of diagnosis. Fungi disease contemplated in the context of the invention include, but are not limited to, Aspergillosis, Black piedra, Candidiasis, Chromomycosis, Cryptococcosis, Onychomycosis, or Otitis externa (otonmycosis), Phaeohyphomycosis, Phycomycosis, Pityriasis versicolor, ringworm, Tinea barbae, Tinea capitis, Tinea corporis, Tinea cruris, Tinea favosa, Tinea imbricata, Tinea manuum, Tinea nigra (palmaris), Tinea pedis, Tinea unguium, Torulopsosis, Trichoniycosis axillaris, White piedra, and their synonyms, to severe systemic or opportunistic infections, such as, but not limited to, Actinomycosis, Aspergilosis, Candidiasis, Chromomycosis, Coccidioidomycosis, Cryptococcosis, Entomophthoramycosis, Geotrichosis, Histoplasmosis, Mucormycosis, Mycetoma, Nocardiosis, North American Blastomycosis, Paracoccidioidomycosis, Phaeohyphomycosis, Phycomycosis, pneumocystic pneumonia, Pythiosis, Sporotrichosis, and Torulopsosis, and their synonyms, some of which may be fatal. Known fungal and mycotic pathogens include, but are not limited to, Absidia spp., Actinomadura madurae, Actinomyces spp., Allescheria boydii, Alternaria spp., Anthopsis deltoidea, Apophysomyces elegans, Arnium leoporinum, Aspergillus spp., Aureobasidium pullulans, Basidiobolus ranarum, Bipolaris spp., Blastomyces dermatitidis, Candida spp., Cephalosporium spp., Chaetoconidium spp., Chaetomium spp., Cladosporium spp., Coccidioides immitis, Conidiobolus spp., Corynebacterium tenuis, Cryptococcus spp., Cunninghamella bertholletiae, Curvularia spp., Dactylaria spp., Epidermophyton spp., Epidermophyton floccosum, Exserophilum spp., Exophiala spp., Fonsecaea spp., Fusarium spp., Geotrichum spp., Helmin thosporium spp., Histoplasma spp., Lecythophora spp., Madurella spp., Malassezia furfur, Microsporum spp., Mucor spp., Mycocentrospora acerina, Nocardia spp., Paracoccidioides brasiliensis, Penicillium spp., Phaeosclera dematioides, Phaeoannellomyces spp., Phialemonium obavatum, Phialophora spp., Phoma spp., Piedraia hortai, Pneumocystis carinii, Pythium insidiosum, Rhinocladiella aquaspersa, Rhizoimucor pusillus, Rhizopus spp., Saksenaea vasiformis, Sarcinomyces phaeomuriformis, Sporothrix schenckii, Syncephalastrum racemosum, Taeniolella boppii, Torulopsosis spp., Trichophyton spp., Trichosporon spp., Ulocladium chartarum, Wangiella dermatitidis, Xylohypha spp., Zygomyetes spp. and their synonyms. Other fungi that have pathogenic potential include, but are not limited to, Thermomucor indicae-sendaticae, Radiomyces spp., and other species of known pathogenic genera.

Other medically relevant microorganisms have been described extensively in the literature, e.g., see Medical Microbiology (1983), the entire contents of which is hereby incorporated by reference.

In other embodiments, the methods and devices described herein can be used for early stage cancer/precancerous detection from circulating extracellular vesicle (EV) encapsulated miRNAs in blood, biofluids, or cerebrospinal fluid (CSF), allowing rapid and accurate diagnosis at a fraction of current cost. The disclosed detection technique is highly sensitive, reliable and requires no amplification, thus removing any bias associated with the amplification process.

The disclosed device is compact, easy to use, low cost, and quick in producing test results from a small sample amount. Hence, the disclosed detection technique and device provide a platform technology applicable to miRNA and DNA biomarkers for various pathogens and cancer types/subtypes.

### Reaction Solutions

Solutions and compositions for a probe-target in hybridization applications are known in the art. Such compositions may comprise, for example, buffering agents, accelerating agents, chelating agents, salts, detergents, and blocking agents. Marangoni flow is relatively easy to achieve in droplets of organic solvents, but can be more challenging in aqueous solutions, such as the type of liquid solutions used in a bioassay including nucleic acid hybridization. To promote Marangoni flow in aqueous droplets, one can use various accelerating additives, such as surfactants and detergents. Other additives can also be added for enhancing product visualization (such as with FRET pairs under a fluorescence microscope) or stabilizing the droplet. Examples may include glycerol, salts (e.g., NaCl and LiCl), and polymers (e.g., FICOLL® copolymer of sucrose and epichlorohydrin),

Example of buffering agents may include SSC, HEPES, SSPE, PIPES, TMAC, TRIS, SET, citric acid, a phosphate buffer, such as, e.g., potassium phosphate or sodium pyrrophosphate, etc. The buffering agents may be present at concentrations from 0.01× to 50×, such as, for example, 0.01×, 0.1×, 0.5×, 1×, 2×, 5×, 10×, 15×, 20×, 25×, 30×, 35×, 40×, 45×, or 50×. Typically, the buffering agents are present at concentrations from 0.1× to 10×.

Example of accelerating agents may include polymers such as FICOLL, PVP, heparin, dextran sulfate, proteins such as BSA, glycols such as ethylene glycol, glycerol, 1,3 propanediol, propylene glycol, or diethylene glycol, combinations thereof such as Dernhardt's solution and BLOTTO, and organic solvents such as formamide, dimethylformamide, DMSO, etc. The accelerating agent may be present at concentrations from 1% to 80% or 0.1× to 10×, such as, for example, 0.1% (or 0.1×), 0.2% (or 0.2×), 0.5% (or 0.5×), 1% (or 1×), 2% (or 2×), 5% (or 5×), 10% (or 10×), 15% (or 15×), 20% (or 20×), 25% (or 25×), 30% (or 30×), 40% (or 40×), 50% (or 50×), 60% (or 60×), 70% (or 70×), or 80% (or 80×). Typically, formamide is present at concentrations from 25% to 75%, such as 25%, 30%, 40%, 50%, 60%, 70%, or 75%, while DMSO, dextran sulfate, and glycol are present at concentrations from 5% to 10%, such as 5%, 6%, 7%, 8%, 9%, or 10%.

Examples of chelating agents may include EDTA, EGTA, etc. The chelating agents may be present at concentrations from 0.1 mM to 10 mM, such as 0.1 mM, 0.2 mM, 0.5 mM, 1 mM, 2 mM, 3 mM, 4 mM, 5 mM, 6 mM, 7 mM, 8 mM, 9 mM, or 10 mM. Typically, the chelating agents are present at concentrations from 0.5 mM to 5 mM, such as 0.5 mM, 1 mM, 1.5 mM, 2 mM, 2.5 mM, 3 mM, 3.5 mM, 4 mM, 4.5 mM, or 5 mM.

Examples of salts may include sodium chloride, sodium phosphate, potassium chloride, potassium phosphate, magnesium chloride, magnesium phosphate, etc. The salts may be present at concentrations from 1 mM to 800 mM, such as 1 mM, 5 mM, 10 mM, 15 mM, 20 mM, 30 mM, 40 mM, 50 mM, 100 mM, 200 mM, 300 mM, 400 mM, 500 mM, 600 mM, 700 mM, or 750 mM. Typically, the salts are present at concentrations from 10 mM to 500 mM, such as 10 mM, 20 mM, 30 mM, 40 mM, 50 mM, 100 mM, 200 mM, 300 mM, 400 mM, or 500 mM.

Examples of detergents may include TWEEN, SDS, TRITON, CHAPS, deoxycholic acid, etc. The detergent may be present at concentrations from 0.001% to 10%, such as, for example, 0.001, 0.01, 0.1, 0.5, 1, 2, 3, 4, 5, 6, 7, 8, 9, or 10%. Typically, the detergents are present at concentrations from 0.01% to 1%, such as 0.01%, 0.02%, 0.03%, 0.05%, 0.1%, 0.2%, 0.3%, 0.4%, 0.5%, 0.6%, 0.7%, 0.8%, 0.9%, or 1%.

Examples of nucleic acid blocking agents may include, for example, yeast tRNA, homopolymer DNA, denatured salmon sperm DNA, herring sperm DNA, total human DNA, COT1 DNA, etc. The blocking nucleic acids may be present at concentrations of 0.05 mg/mL to 100 mg/mL. However, the compositions and methods of the invention surprisingly show significantly reduced background levels without the need for blocking agents.

The compositions described above may comprise any of the components recited above in combination with at least one polar aprotic solvent. The components may be present at the same concentrations as used in traditional denaturing solutions, or may be present at higher or lower concentrations, or may be omitted completely. In some embodiments, as sessile droplets provide benefits of internal currents and decreasing volume when evaporating that allow for enhanced molecular interactions, one or more of the components may be applied to the substrate or reaction area at concentrations much lower.

The solution may have some salt/sugar present to shield non-specific interactions, but the concentration may be low enough that full evaporation of the droplet does not cause sufficient crystallization to compromise the assay. For example, Na+ of concentrations ≤50 mM may be sufficient to screen non-specific interactions and not cause detrimental crystallization upon full evaporation of a drop. A NaCl concentration of 50 mM would also be sufficient to create the flow patterns in a drop of liquid carrier evaporated on PDMS.

### Kits

In another aspect, this disclosure provides a kit that comprise various reagents useful for carrying out the method described herein. The kit may comprise suitably substrates in derivatized or underivatized form, and may also include reagents for sample isolation, purification or preparation, probes, hybridization solutions, liquid carriers, hygroscopic materials, salts, wash solutions, blocking agents, HCT reaction mixtures, probes, reporter molecules, means for detecting the probes or reporter molecules.

The components of the kits may be packaged either in aqueous media or in lyophilized form. When reagents and/or components are provided as a dry powder, the powder can be reconstituted by the addition of a suitable solvent. It is envisioned that the solvent may also be provided.

The container means of the kits will generally include at least one vial, test tube, flask, bottle, syringe or other container means, into which a component may be placed, and preferably, suitably aliquoted. Where there is more than one component in the kit, the kit also will generally contain a second, third or other additional containers into which the additional components may be separately placed. However, various combinations of components may be comprised in a vial. The kits of the present invention also will typically include a means for containing the reagent vials and other kit components in close confinement for commercial sale. Such containers may include injection or blow molded plastic containers into which the desired vials are retained.

Irrespective of the number or type of containers, the kits of the invention may also comprise, or be packaged with, instructions for use of the various reagents. In some embodiments, the kit also includes a container that contains the composition and optionally informational material. The informational material can be descriptive, instructional, marketing or other material that relates to the methods described herein and/or the use of the agents. The informational material of the kits is not limited in its form. In some embodiments, the informational material can include information about production of the composition, concentration, date of expiration, batch or production site information, and so forth. The information can be provided in a variety of formats, including printed text, computer-readable material, video recording, or audio recording, or information that contains a link or address to substantive material. The kit optionally includes a device suitable for carrying the methods disclosed herein.

### Definition

As used herein, the term “drop” or “droplet” refers to a small volume of liquid that is immiscible with its surroundings (e.g., gases, liquids, surfaces, etc.). A droplet may reside upon a surface, be encapsulated by a fluid with which it is immiscible (e.g., the continuous phase of an emulsion, a gas (e.g., air, nitrogen)), or a combination thereof. A droplet is typically spherical or substantially spherical in shape, but may be non-spherical. The shape of an otherwise spherical or substantially spherical droplet may be altered by deposition onto a surface or constriction in a capillary channel of smaller diameter. A droplet may be a “simple droplet” or a “compound droplet,” wherein one droplet encapsulates one or more additional smaller droplets. The volume of a droplet and/or the average volume of a set of droplets provided herein is typically less than about one hundred microliters (e.g. 100 μL, 10 L, 1 μL, 100 nL, 10 nL, 1 nL, 100 pL, 10 pL, 1 6 L, 100 fL, 10 fL, or 1 fL). The diameter of a droplet and/or the average diameter of a set of droplets provided herein is typically less than about one millimeter (e.g. 1 mm, 100 μm, 10 μm, or 1 μm). Droplets may be formed by any suitable technique (e.g., emulsification, microfluidics, injection etc.) and may be monodisperse (e.g., substantially monodisperse) or polydisperse.

A sessile droplet refers to a droplet that resides upon a surface of a solid or semisolid substrate.

An “aqueous solution” is to be understood as a solution containing water, even small amounts of water. For example, a solution containing 1% water is to be understood as an aqueous solution.

“Hybridization reaction,” “hybridization assay,” “hybridization experiment,” “hybridization procedure,” “hybridization technique,” “hybridization method,” etc. are to be understood as referring to any process that involves hybridization of nucleic acids. Unless otherwise specified, the terms “hybridization” and “hybridization step” are to be understood as referring to the re-annealing step of the hybridization procedure as well as the denaturation step (if present).

A “reaction solution” refers to a solution or composition for performing a reaction. When the reaction involves detection or hybridization, the reaction solution is also referred as a “detection solution,” (i.e., a solution or composition for performing a reaction such as a detection assay) or a hybridization solution (i.e., a solution or composition for performing a reaction such as a hybridization assay).

“Hybridization solution” refers to an aqueous solution for use in a hybridization composition. Hybridization solutions are discussed herein and may comprise, e.g., buffering agents, accelerating agents, chelating agents, salts, detergents, and blocking agents.

A “Hybridization composition” refers to an aqueous solution for performing a hybridization procedure, for example, to bind a probe to a nucleic acid sequence.

Hybridization compositions may comprise, e.g., at least one polar aprotic solvent, at least one nucleic acid sequence, and a hybridization solution. Hybridization compositions do not comprise enzymes or other components, such as deoxynucleoside triphosphates (dNTPs), for amplifying nucleic acids in a biological sample.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C.

A nucleic acid or polynucleotide refers to a DNA molecule (for example, but not limited to, a cDNA or genomic DNA) or an RNA molecule (for example, but not limited to, an mRNA, rRNA, tRNA, miRNA, or siRNA), and includes DNA or RNA analogs. A DNA or RNA analog can be synthesized from nucleotide analogs. The DNA or RNA molecules may include portions that are not naturally occurring, such as modified bases, modified backbone, deoxyribonucleotides in an RNA, etc. The nucleic acid molecule can be single-stranded or double-stranded.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

### EXAMPLES

### Example 1

This example descibes materials and methods used in Examples 2-7 bellow

### Materials

All DNA oligos were purchased from IDT Integrated DNA Technology (Coralville, IA, USA). All 3D printed materials were designed in SOLIDWORKS 2018-2019 Student Edition and printed using polylactic acid (PLA) and a Prusa i3 MK3S purchased from Prusa (Prague, Czech Republic). Droplets were evaporated using Drierite from W. A. Hammond Drierite Co. (Xenia, OH, USA). Gel electrophoresis was performed using HE33 Mini Horizontal Agarose Electrophoresis Unit and MIGHTY SLIM SX250 Power Supply from Hoefer (Holliston, MA, USA). UltraPure™ Agarose, Sybr Safe DNA Gel Stain, sodium chloride, magnesium chloride, sodium citrate, and an Amplitron® II thermocycler were purchased from Thermo Fisher Scientific (Waltham, MA, USA). Water was purified using Barnstead™ NANOpure II from Thermo Fisher Scientific. 10× tris borate EDTA (TBE) was purchased from Bio-Rad Laboratories (Hercules, CA, USA) as well as the CHEMIDOC MP used to image the agarose gels. Sylgard™ 184 Silicone Elastomer Kit was purchased from Dow (Midland, MI, USA). A FLUOROMAX-4 Spectrofluorometer from Horiba Scientific (Piscataway, NJ, USA) was used for obtaining spectral data. A UV-1800 spectrophotometer by Shimadzu (Kyoto, Japan) was used for the DNA hairpin melt analysis. The data was analyzed using JMP Pro 16.

### DNA Hairpin Annealing

H1 and H2 DNA hairpins were incubated in a thermocycler at 95° C. for 5 minutes and then cooled to room temperature over 30 minutes.

### Pedestal Fabrication

The 1.5 mm pedestal ring molds and drying chamber were designed using computer software and 3D printed. A silicone elastomer kit was mixed at a 7:1 ratio (part A to part B) and poured into the pedestal molds to create the polydimethylsiloxane (PDMS) pedestals. PDMS in molds were degassed and left to cure overnight. Cured PDMS pedestals were removed from molds and placed in the drying chamber.

### HCR Preparation

Annealed H1 and H2 DNA hairpins were diluted to 1500 nM concentrations and initiator was diluted to 3× its final concentration in the defined assay buffers. For fluorescence assays, the H1 and H2 have a 3′ ATTO 550N fluorophore and 5′ ATTO 647N fluorophore conjugation, respectively. All buffers were created by diluting 5× saline-sodium citrate (SSC) (750 mM sodium chloride and 75 mM sodium citrate) in water and adding magnesium chloride when indicated. Diluted hairpins and initiator were then mixed in a 0.5 μL cuvette at a 1:1:1 ratio. Final concentrations for H1 and H2 were 500 nM, and initiator concentrations were as indicated in the resulting figures. Two 5 μL droplets were pipetted on to the pedestals for each reaction, and 10 μL of each reaction were left in the cuvette as the liquid incubation control. Desiccant was added to the drying chamber and the experiment was covered with the chamber lid and left to evaporate completely over 1 hour. 5 μL of water was pipetted onto each dried sessile droplet pedestal and left covered to resuspend the dried material. After 5 minutes, the droplets were drawn into a pipette and added to a cuvette, two droplets (two separate pedestals) per reaction for a combined final volume of 10 μL per reaction.

### Agarose Gel Electrophoresis

3% agarose gels were prepared by boiling 1.2 grams agarose in 40 mL 1×TBE via microwave. 4 μL SYBR SAFE was added to the molten solution and mixed thoroughly before pouring into the gel mold to set. 10 μL of sample was prepared for gel electrophoresis by adding 3.5 μL of water and 1.5 μL of an aqueous 40% sucrose solution prior to running in the agarose gel in 0.5×TBE running buffer at 150 volts for 40 minutes.

### Förster Resonance Energy Transfer (FRET)

For the fluorescence HCR assay, 10 μL of sample was diluted in 690 μL of water. The 700 μL dilution was added to a quartz cuvette and the spectral emission intensities were gathered by a spectrofluorometer by exciting the sample at a 560 nm wavelength and scanning emission intensities from 567 nm to 750 nm. Spectral intensities were normalized to the peak emission intensity and graphed using MATLAB R2020b.

### Example 2

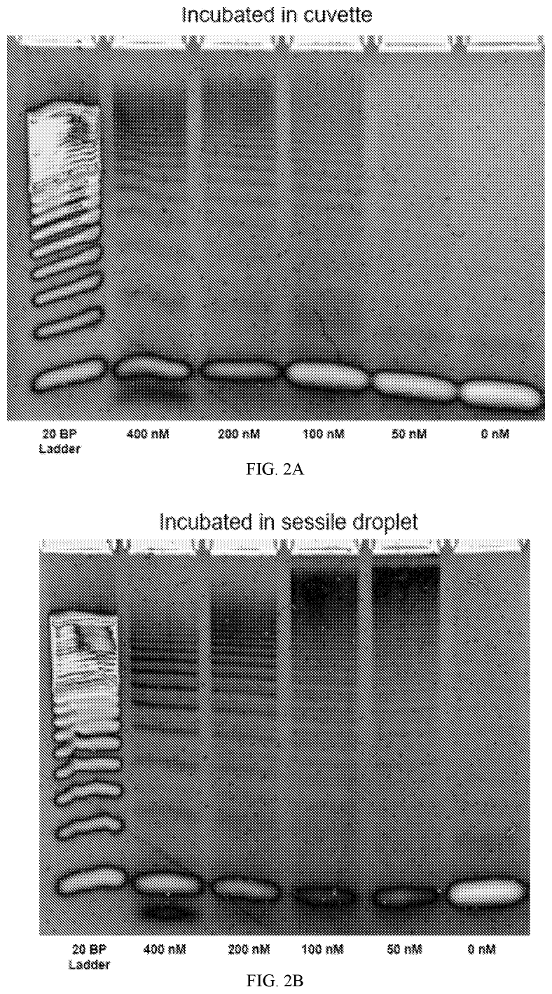

In this example, HCR was used to demonstrate the significant improvement in extended DNA hairpin hybridization while incubated in a sessile droplet versus traditional liquid cuvette incubation. Briefly, HCR assays were carried out in both sessile droplets (S) and cuvettes in the manner described above. The resulting hybridization products were visualized and examined in the manner described above. The results are shown in FIGS. 2, 3, and 4.

As demonstrated in FIGS. 2A and 2B, significantly more HCR product was formed by the hairpin probes when incubated in a sessile droplet when compared to traditional incubation conditions in a cuvette, particularly at lower trigger concentrations (50 nM). Both experiments in FIGS. 2A and B were prepared in an identical manner using 0.5×SSC buffer with 10 mM MgCl2.

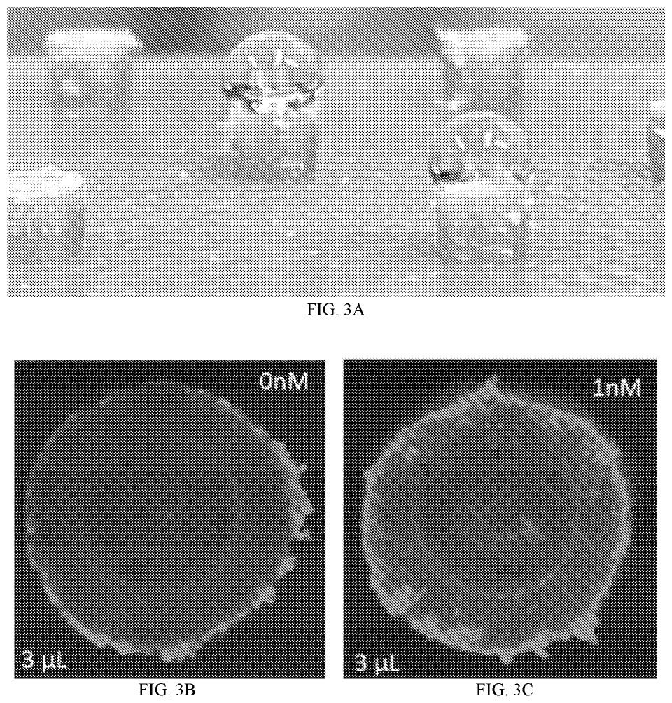

FIG. 3A shows a custom designed and 3D printed array of 2 mm diameter pedestals used to dry the HCR sessile droplets. Fluorescence images of the resulting FRET from the HCR product are shown in FIGS. 3B and C.

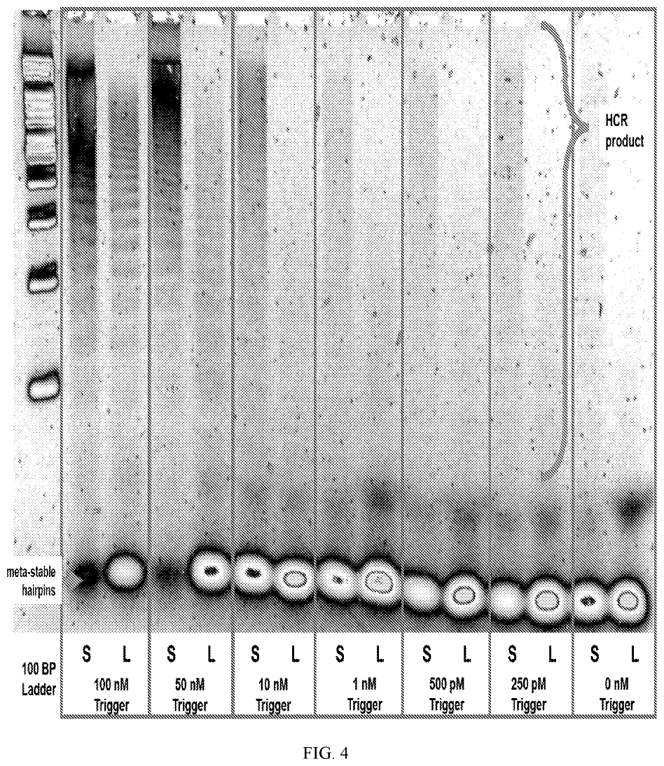

FIG. 4 shows a direct comparison between HCR experiments incubated in sessile droplets (S) and cuvettes (L) with varying concentrations of trigger sequences. Each sessile droplet lane had distinctly higher molecular weight banding with greater intensity over its liquid cuvette compliment lane, demonstrating an increased hybridization activity due to sessile droplet incubation.

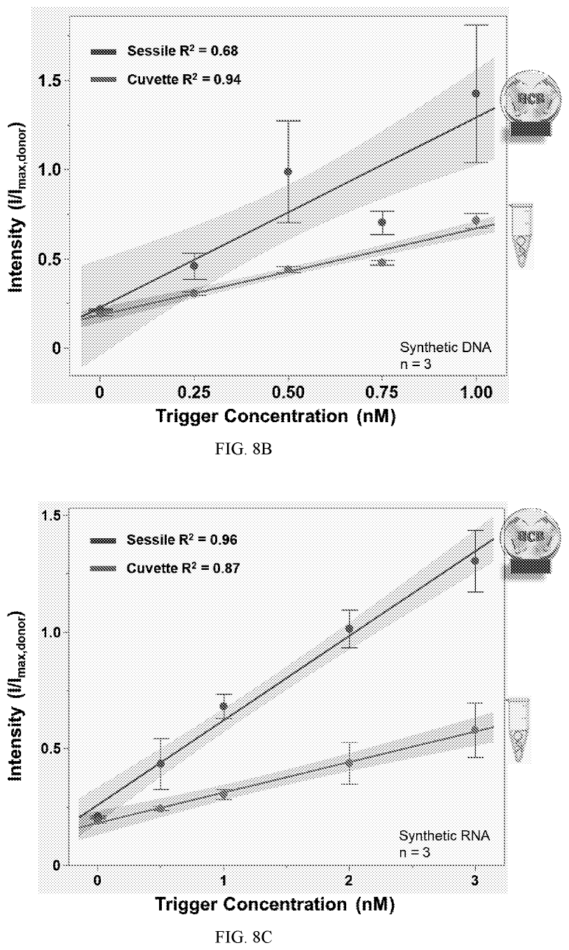

Additional results are sown in FIGS. 8A, 8B, and 8C. The agarose gel results shown in FIG. 8A empirically indicated improved hybridization of HCR through sessile droplet incubation when compared to traditional cuvette incubation. It was also found addition (+) of Tween 20 detergent to the hybridization buffer improved HCR product in both sessile and cuvette incubations over hybridization buffer without (−) Tween 20. The improved hybridization of HCR through sessile droplet incubation was demonstrated by spectrofluorometer data as shown in FIG. 8B and FIG. 8C where FRET intensities of HCR product from synthetic proviral DNA target (FIG. 8B) and synthetic viral RNA target (FIG. 8C) were normalized using the FRET ratio.

### Example 3

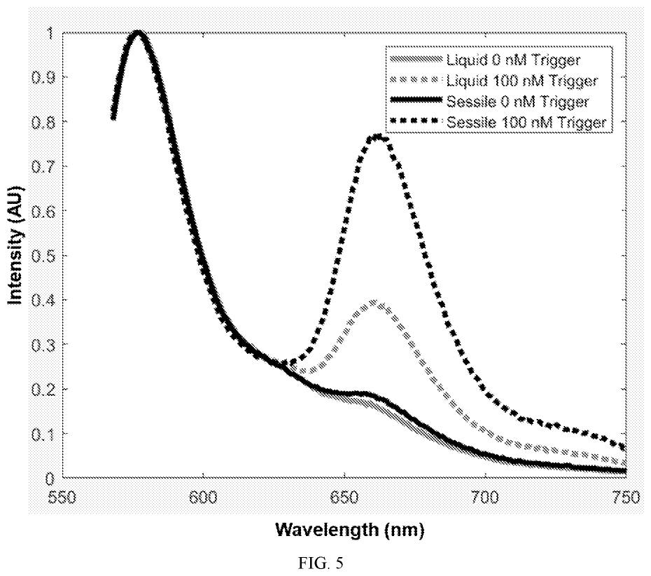

In this example, inventors further demonstrated the sessile droplet induced hybridization effect through FRET-HCR. Increased hybridization results in more H1 and H2 FRET pairs within transmittable distance to each other. More specifically, when excited at the donor fluorophore excitation wavelength (560 nm), hybridized H1 fluorophores transmitted resonance energy to the hybridized H2 fluorophores, emitting fluorescence at a much longer wavelength (˜664 nm) than an unhybridized H1 fluorophore would emit. As shown in FIG. 5, the 100 nM trigger sessile droplet experiment had a significantly higher H2 intensity over the 100 nM trigger liquid cuvette experiment, indicating a proportional increase in HCR hybridization due to sessile incubation.

### Example 4

In this example, assays were carried out to examine effects of buffer ion concentration on hybridization efficiency. Buffers containing high sodium and magnesium concentrations are known to improve DNA hybridization. However, as the volume of the evaporating sessile droplet decreases, the ion concentration of the buffer increases, affecting the hybridization of the DNA oligos. Inventors used varying concentrations of SSC buffer with and without magnesium chloride to demonstrate the impact of ions on sessile droplet enhanced hybridization. A 5× concentration of SSC is commonly used for hybridization experiments. However, as shown in FIG. 6, while the highest ion concentration buffer (0.5×SSC with 10 mM MgCl₂) had the greatest HCR hybridization product, it also has significantly more uninitiated hybridization in its 0 nM control lane than the lower ion buffers. The 0.25×SSC with 10 mM MgCl₂ appeared to have high molecular weight banding at 500 pM trigger concentration with significantly less banding in its 0 nM control lane, suggesting that lower salt concentration buffers may have beneficial impacts on sessile droplet hybridization assays.

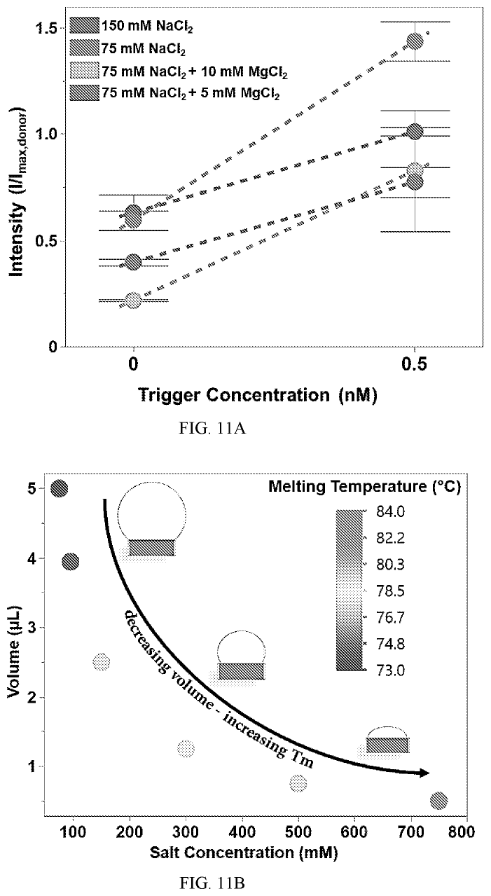

Similar assays were carried out in the presence of different concentrations of NaCl or MgCl₂, or both to examine effects of ion concentration on hybridization efficiency. The results are shown in FIG. 11A. As shown in the figure, higher ion concentration decreased overall hybridization.

Additional assays were carried out to examine experimental melt temperature of the H1 hairpin at different sodium concentrations. The results are shown in FIG. 11B. As the salt concentration increases the melting temperature of the hairpin increases; shown here as the sessile droplet decreasing in volume due to evaporation. The volume to salt concentration was calculated using 75 mM NaCl as the starting concentration. The increasing stability of the hairpin by increasing salt concentration supports the results in FIG. 11A, indicating higher salt concentration can decrease hybridization. Salt concentration in the buffer will increase as the droplet evaporates, highlighting the importance of buffer composition during sessile droplet incubation. All buffers in FIG. 11A contained 0.1% Tween 20. Buffers in FIG. 11B do not contain Tween 20 because excessive micelle formation would disrupt the UV/vis data. Data was analyzed using JMP Pro 16.

### Example 5

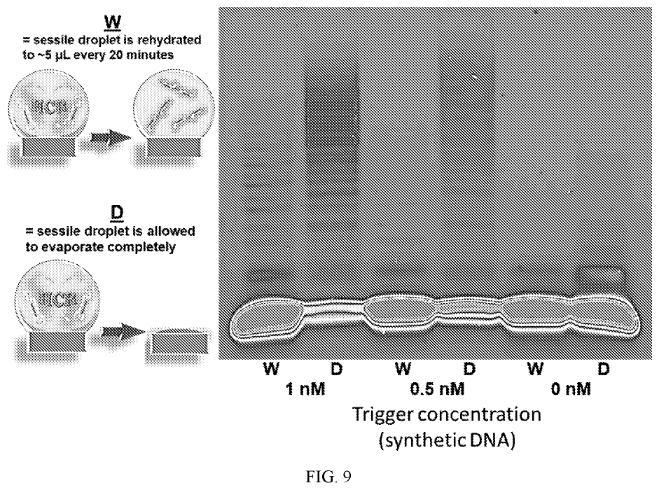

HCR assays were carried out against synthetic DNA trigger in two different conditions. In the first, “wet” condition (W), the sessile droplet was rehydrated every 20 minutes to keep the original volume of 5 μL. In the second “dry” condition (D), the sessile droplet was allowed to evaporate fully. Both droplets were incubated for 70 minutes before being analyzed by agarose gel. The results as shown in FIG. 9 indicate a significant increase in HCR product in the dry condition (D), demonstrating the importance of decreasing volume in the sessile droplet.

### Example 6

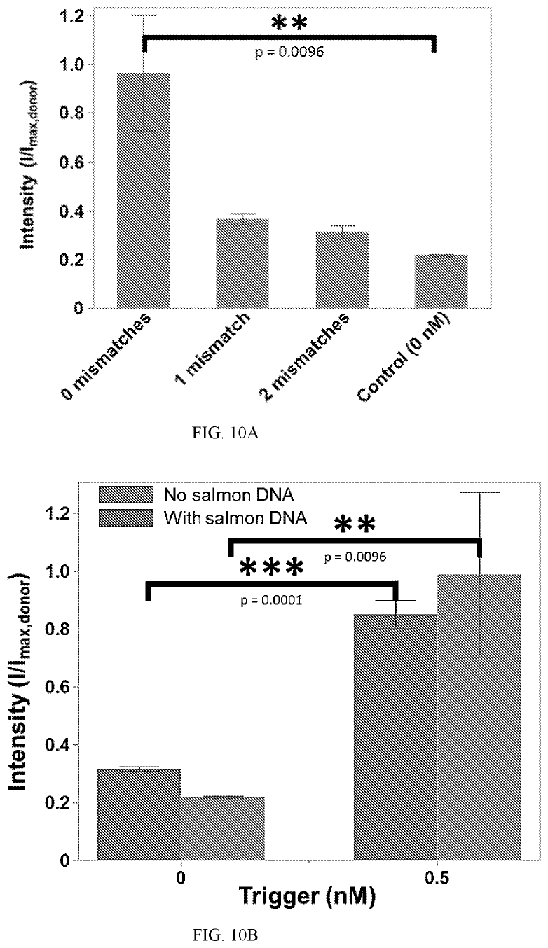

The HCR sessile droplet assays described herein were carried out in presence of the following three synthetic DNA triggers having 0, 1 and 2 mismatches: AGGTTTGGGGAAGAGACA (SEQ ID NO: 1), AGGTTTGGGGAGGAGACA (SEQ ID NO: 2), and AGGTCTGGGGTAGAGACA (SEQ ID NO: 3). The results are shown in FIG. 10A. It was found that there was no significant increase in HCR product with 1 and 2 mismatches over the 0 nM trigger control. This result demonstrates high specificity of the HCR sessile droplet assays.

The HCR sessile droplet assays described herein were carried out in the presence of non-specific DNA such as salmon DNA at a 5:1 mass ratio of salmon DNA to synthetic trigger DNA. All data was analyzed using JMP Pro 16, and p values calculated using a two-tailed Student's t-test. The results are shown in FIG. 10B. As shown in FIG. 10B, HCR sessile droplet assays demonstrate a high resistance to off-target hybridization.

### Example 7

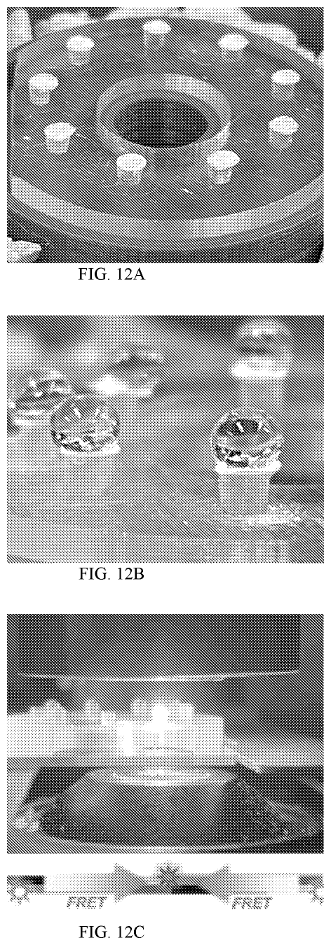

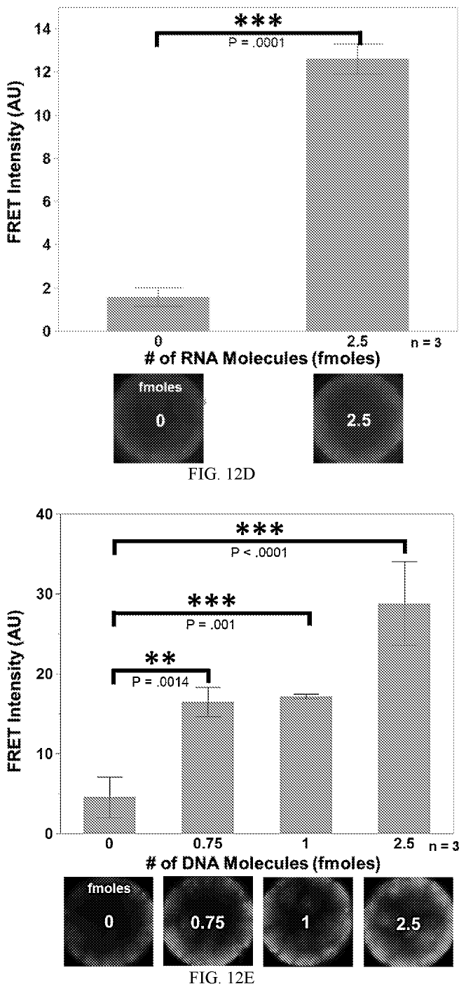

The HCR sessile droplet assays described herein were carried out to detect synthetic HIV RNA and proviral DNA directly on filter paper (Fusion 5, Cytiva). The setups of the assays are shown in FIGS. 12A, 12B, and 12C. Briefly, 1.5 mm diameter pads were cut and adhered to PDMS pedestals to allow for sessile droplet formation on the hydrophilic material (FIG. 12A). Target nucleic acids were dried onto the paper disks. Then, 5 μL of HCR solution (H1 and H2) were applied to each pedestal as a sessile droplet and allowed to dry completely (FIG. 12B). The dried paper was then imaged using a fluorescence microscope with a FRET cube (FIG. 12C). Image intensities were analyzed using ImageJ. P values were calculated using a two-tailed Student's t-test. The results are shown in FIGS. 12D and 12E. As shown in the figures, the assays successfully detected as little as 2.5 fmoles of synthetic HIV RNA and 0.75 fmoles of proviral DNA.

### REFERENCES

1. Luo J, Xu Y, Huang J, Zhang S, Xu Q, He J. Enzyme-free amplified detection of circulating microRNA by making use of DNA circuits, a DNAzyme, and a catalytic hairpin assembly. Microchim Acta 2017 1851. 2017; 185(1):1-6. doi:10.1007/500604-017-2565-9
2. Guixiu Dong, Jianyuan Dai, Limin Jin, et al. A rapid room-temperature DNA amplification and detection strategy based on nicking endonuclease and catalyzed hairpin assembly. Anal Methods. 2019; 11(19):2537-2541. doi:10.1039/C9AY00507B
3. Dong Q, Liu Q, Guo L, et al. A signal-flexible gene diagnostic strategy coupling loop-mediated isothermal amplification with hybridization chain reaction. Anal Chim Acta. 2019; 1079:171-179. doi:10.1016/j.aca.2019.06.048
4. Wang W, Nie A, Lu Z, Li J, Shu M, Han H. Catalytic hairpin assembly-assisted lateral flow assay for visual determination of microRNA-21 using gold nanoparticles. Microchim Acta. 2019; 186(9):1-9. doi:10.1007/s00604-019-3743-8
5. Wang W-J, Li J-J, Rui K, Gai P-P, Zhang J-R, Zhu J-J. Sensitive Electrochemical Detection of Telomerase Activity Using Spherical Nucleic Acids Gold Nanoparticles Triggered Mimic-Hybridization Chain Reaction Enzyme-Free Dual Signal Amplification. Anal Chem. 2015; 87(5):3019-3026. doi:10.1021/AC504652E
6. Hernandez-Perez R, Fan Z H, Garcia-Cordero JL. Evaporation-Driven Bioassays in Suspended Droplets. Anal Chem. 2016; 88(14):7312-7317. doi:10.1021/acs.analchem.6b01657
7. Hu H, Larson RG. Marangoni effect reverses coffee-ring depositions. J Phys Chem B. 2006; 110(14):7090-7094. doi:10.1021/jp0609232
8. Mitre E, Schulze M, Cumme GA, Röβler F, Rausch T, Rhode H. Turbo-Mixing in Microplates. J Biomol Screen. 2007; 12(3):361-369. doi:10.1177/1087057106297565.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method for detecting a target nucleic acid in a test sample, comprising
providing a substrate having a reaction area deposited thereon with the test sample and a detection solution to form a reaction droplet, said detection solution comprising a detection agent having a sequence that is complementary to a strand of the target nucleic acid;
incubating the reaction droplet under conditions allowing evaporation thereof, and detecting a hybridization complex of the target nucleic acid and the sequence in the reaction droplet, whereby a presence of the hybridization complex indicates presence of the target nucleic acid in the test sample.

2. The method of claim 1, wherein the providing step comprises
placing a sample droplet comprising the test sample onto the reaction area and
contacting the reaction area or the test sample thereon with the detection solution.

3. The method of claim 2, further comprising drying the sample droplet on the reaction area before the contacting step.

4. The method of claim 1, wherein the reaction area is hydrophilic and surrounded by a hydrophobic area.

5. The method of claim 1, wherein the reaction area or the substrate is surrounded by a non-permeable boundary.

6. The method of claim 1, wherein the reaction area or the substrate comprises a material selected from the group consisting of glass, plastic, metal, silicon, and paper.

7. The method of claim 1, wherein the substrate comprises a structure of a pilar or a pedestal having a circular top that serves as the reaction area.

8. The method of claim 7, wherein the circular top has a diameter of about 0.5 μm to 4 mm.

9. The method of claim 1, wherein the reaction droplet or the sample droplet is about 1-25 μl.

10. The method of claim 1, wherein the detection solution comprises a hybridization chain reaction (HCR) mixture.

11. The method of claim 1, wherein the detection solution comprises about 15 to about 800 mM concentration of any combination of Na and K ions.

12. The method of claim 11, wherein the detection solution comprises one or more of about 0.1 to about 5.0×SSC, about 1 to about 15 mM Mg2+, PBS, TBS, a detergent, glycerol, and sucrose.

13. The method of claim 1, wherein the conditions include
a humidity of about 5% to about 95%,
a temperature of about 10 to about 50° C., and/or
a time duration of about 10 to about 300 minutes.

14. The method of claim 1, wherein the target nucleic acid is a DNA or a RNA.

15. The method of claim 1, wherein the target nucleic acid is a nucleic acid of a pathogen.

16. The method of claim 15, wherein the pathogen is a virus.

17. The method of claim 16, wherein the virus is HIV, Dengue, SARS-CoV-2, or Ebola.

18. The method of claim 1, wherein the test sample comprises a blood sample, a sputum sample, a urine sample, a urinary swab sample, or a saliva sample.

19. A kit comprising the substrate and the detection solution as defined in claim 1.
